(19) United States
(12) Patent Application Publication
(10) Pub. No.: US 2003/0170198 A1
(43) Pub. Date: Sep. 11, 2003

(54) Transparent shaving gel

(76) Inventor: Brian W. Williams, Grayson, GA (US)

Correspondence Address: BRIAN WILLIAMS, 2490 TRIBBLE CREEK COVE, GRAYSON, GA, 30017, US

(21) Appl. No.: 10/090,960

(22) Filed: Mar. 5, 2002

(51) Int. Cl. A61K 7/15

(52) U.S. Cl. 424/73

(57) ABSTRACT

A transparent shaving gel is disclosed comprising of in percent by weight with the balance being water, about 20% of a humectant(s) such as Glycerin, Propylene Glycol, and/or Glycereth-26. About 5% of a lubricant(s)/skin conditioners(s) such as Aloe Vera gel, vitamin “E” oil and/or PEG 600. About 0.8% of a gellant such as carbomers (such as Carbopol 940 available from BF Goodrich), hydroethylcellulose, or hydropropylcellulose. About 0.8% of a neutralizer such as Triethanolamine (TEA), Sodium hydroxide, or Potassium hydroxide, and about 0.5% of a preservative(s) such as Methylparaben, Propylparaben, and/or DMDM Hydantoin (such as Glydant available from Lonza).

### CROSS REFERENCE TO RELATED PATENTS

| | | |
|---|---|---|
| U.S. Patent # 5,340,571 | 08/1994 Grace | 424/73 |
| U.S. Patent # 6,024,951 | 02/2000 Babinski et al | 424/73 |
| U.S. Patent # 5,849,281 | 12/1998 Babinski et al | 424/73 |
| U.S. Patent # 5,888,490 | 03/1999 Hall-Puzio | 424/73 |
| U.S. Patent # 5,560,859 | 10/1996 Hartmann et al | 252/90 |

### FEDERALLY SPONSORED RESEARCH OR DEVELOPEMENT

N/A

### BACKGROUND OF INVENTION

N/A

There are many shaving gel compositions available to consumers today. These are applied to the skin typically with the fingers, worked into a later that is then removed typically with a razor. The purpose of the present invention is to provide a unique shaving gel in that it remains transparent when applied to the skin. This will allow for visualization of the hairline while shaving, thereby facilitating a more accurate trace of the hairline, ultimately resulting in a faster shave, and more aesthetic post shave look.

### BRIEF SUMMARY OF INVENTION

The present invention relates a shaving gel comprising of in percent by weight with the balance being water, about 20% of a humectant(s) such as Glycerin, Propylene Glycol, and/or Glycereth-26. About 5% of a lubricant(s)/skin conditioners(s) such as Aloe Vera gel, vitamin “E” oil and/or PEG 600. About 0.8% of a gellant such as carbomers (such as Carbopol 940 available from BF Goodrich), hydroethylcellulose, or hydropropylcellulose. About 0.8% of a neutralizer such as Triethanolamine (TEA), Sodium hydroxide, or Potassium hydroxide, and about 0.5% of a preservative(s) such as Methylparaben, Propylparaben, and/or DMDM Hydantoin (such as Glydant available from Lonza).

### DESCRIPTION OF SEVERAL VIEWS OF THE DRAWING

N/A

### DETAILED DESCRIPTION OF INVENTION

The related shaving composition will have the following formulae wherein the percentages are by weight:

| Ingredient | % |
|---|---|
| Glycerin | 8-10 |
| Propylene Glycol | 8-10 |
| PEG 600 | 1-2 |
| Aloe Vera gel | 1-2 |
| Vitamin “E” oil | 1 |
| Carbopol 940 | 0.5-0.8 |
| TEA | 0.5-0.8 |
| Methylparaben | 0.2-0.5 |
| Propylparaben Glycol | 0.2-0.5 |
| Balance deionized water | |

Fragrances and tints may be added as deemed necessary, further, one or more of the components may be substituted with equivalent ingredients. However, it will be understood that the premise of the composition is to remain transparent when applied to the skin and no ingredients that lather, or in any other way obscure the view of the hairline, will be used.

The formulation of the present invention is prepared by (A) sifting the Carbopol 940 into the vortex of rapidly agitating room temperature deiomzed water. Continue agitation until uniform dispersion is produced. Add glycerin with moderate agitation. (B) In a side vessel blend parabens, Propylene Glycol, and Peg 600 using heat (50%-55° C.) until clear. Incorporate Aloe Vera gel and vitamin “E” oil, cool mixture to 45° C. Slowly add solution (B) to solution (A) with moderate agitation. Add TEA with sweeping agitation until a clear uniform gel is obtained.

What I claim is:

1. A transparent shaving gel composition consisting essentially of in percent by weight with the balance being water;
(a) About 15 to 20% of a humectant such as propylene glycol, glycerin, and/or glycereth-26.
(b) About 2 to 5% of a lubricant/skin conditioner(s) such as aloe vera gel, vitamin “E” oil and/or Peg 600.
(c) About 0.5-0.8% of a gellant such as carbomers (such as Carbopol 940 available from BF Goodrich), hydroethylcellulose, or hydropropylcellulose.
(d) About 0.5-0.8% of a neutralizer such as TEA, Sodium hydroxide, or Potassium hydroxide.
(e) About 0.2-0.5% of a preservative such as Methylparaben, Propylparaben, and/or DMDM Hydantoin (such as Glydant available from Lonza).
